(19) United States
(12) Patent Application Publication
(10) Pub. No.: US 2023/0264950 A1
(43) Pub. Date: Aug. 24, 2023

(54) DEVICE AND METHOD FOR HYBRID PRODUCTION OF SYNTHETIC DIHYDROGEN AND/OR SYNTHETIC METHAN

(71) Applicant: ENGIE, Courbevoie (FR)

(72) Inventors: Yilmaz KARA, Courbevoie (FR); Stéphane FORTIN, Courbevoie (FR); Maxime HERVY, Courbevoie (FR); Jonathan MAISTRELLO, Courbevoie (FR)

(21) Appl. No.: 18/005,607

(22) PCT Filed: Jul. 13, 2021
(86) PCT No.: PCT/EP2021/069502

§ 371 Date: Jan. 15, 2023

(30) Foreign Application Priority Data
Jul. 14, 2020 (WO) PCT/FR2020/051265
Aug. 5, 2020 (FR) FR2008307
Apr. 21, 2021 (FR) FR2104177

(51) Int. Cl. C01B 3/16; B01J 8/18; C07C 1/04; B01J 8/24

(52) U.S. Cl. CPC C01B 3/16; B01J 8/1809; C07C 1/041; B01J 8/24; C01B 2203/062; C01B 2203/16; C01B 2203/0405; C01B 2203/0415; C01B 2203/043; C01B 2203/046; C01B 2203/0475; C07C 2521/04; C07C 2523/755

(57) ABSTRACT

The device (100) for hybrid production of synthetic dihydrogen and/or synthetic methane comprises:
an inlet (105) for a synthesis gas stream preferably comprising at least CO and H₂,
a catalytic conversion reactor (110), the following alternative configurations:
   a first configuration in which the operating conditions of the reactor promote a Sabatier reaction, so as to produce an outlet gas comprising mainly methane, or
   a second configuration in which the operating conditions of the reactor promote a water gas shift reaction, so as to produce an outlet gas comprising mainly dihydrogen;
an outlet (115) for synthetic dihydrogen and/or synthetic methane and
a control system (120) comprising a means (121) for selecting a configuration for operating the reactor and a control means (122) according to the selected configuration, the reactor being configured to operate according to a command.

### TECHNICAL FIELD OF THE INVENTION

The present invention relates to a device for the hybrid production of synthetic dihydrogen and/or synthetic natural gas, here also called synthetic methane, and a method for the hybrid production of synthetic dihydrogen and/or synthetic methane. It applies, in particular, to the field of waste and biomass recovery. This invention can also be applied to a synthetic gas produced from the conversion of carbon or any other hydrocarbon materials or any gas containing at least carbon monoxide (CO).

### STATE OF THE ART

In the fight against climate change and the reduction in greenhouse gas emissions, the production of energy from biomass and waste or from a synthetic gas produced from the conversion of carbon or any other hydrocarbon materials or any gas containing at least CO, is a vital alternative.

Carbon neutral, or almost carbon neutral (for waste that is often not 100% biogenic, because it usually contains fossil plastics for example), these solutions make it possible to produce numerous energy carriers (electricity, heat, liquid biofuels, chemical products, biomethane, hydrogen, etc.) by integrating a circular economy approach. From low (<2 MWth) to high capacity (>100 MWth), these processes can also provide delocalised waste recovery solutions.

Biomethane and bio-hydrogen (hereinafter alternatively “biohydrogen”, “hydrogen” or “dihydrogen”) are destined to play a major role in the global energy mix, with biomethane replacing natural gas and bio-hydrogen replacing the hydrogen mainly produced from reforming natural gas and, to a lesser extent, from the electrolysis of water. In addition, the expected emergence of mobility means using these two energy carriers could result in a significant increase in demand. At this time, the biomethane market is clearly established. However, the demand for bio-hydrogen in the coming years is uncertain since it is dependent on many elements, including the creation of distribution networks and the mass development, for example, of hydrogen mobility.

Many processes and systems have been developed to separately produce either methane or hydrogen from carbon materials. However, none of these systems make it possible to:
adapt its production (biomethane or bio-hydrogen) according to the market needs, and therefore boost the installation of these production plants, which will provide the ability to adapt responsively;
mainly produce biomethane while occasionally producing hydrogen to feed the small hydrogen stations;
mainly produce hydrogen and occasionally produce biomethane according to fluctuations in the demand for hydrogen; and
quickly switch from methane production to hydrogen production, and vice versa.

Methanation consists of converting the carbon monoxide or dioxide in the presence of hydrogen and a catalyst or a biological strain to produce methane. It is governed by the following competitive hydrogenation reactions:

CO+3H2⇄CH₄+H₂O ΔG298K=−206 kJ/mol (R2—méthanation CO)

CO₂+4H₂⇄CH₄+2H₂O ΔG298K=−165 kJ/mol (R3—méthanation CO₂) [Formula 1]

In the conditions generally used to produce SNG (“Synthesis Natural Gas”) from syngas produced by pyrogasification, hereinafter referred to alternatively as “gasification”, “pyrolysis” or “pyrogasification”, the CO methanation reaction (R2) is strongly encouraged because in most cases of the sub-stoichiometry in hydrogen.

The methanation reaction is a strongly exothermic reaction with a reduction in the number of moles; as per Le Chatelier's principle, the reaction is encouraged by increasing the pressure and discouraged by increasing the temperature.

The production of methane from CO and H2 is optimum for a gas with a composition close to the stoichiometric composition, i.e. when the H₂/CO ratio is close to 3. The syngas produced by vapour gasification, in particular from biomass, is characterised by a lower H₂/CO ratio, of the order of 1 to 2 when the proportion of vapour to biomass on input to gasification is below 1, which is the most common case in the state of the art. Also, this ratio must be adjusted to maximise the production of methane, by adding hydrogen, for example, from an unavoidable source or produced by electrolysis of water, or most often by producing the hydrogen by the reaction between carbon monoxide and water, through the Water Gas Shift reaction (R1), referred to as “WGS”:

CO+H₂O⇄H₂+CO₂ ΔG298K=−41 kJ/mol (R1—Water Gas Shift) [Formula 2]

The WGS reaction can be carried out in a specific reactor placed upstream from the methanation. However, in the case of certain processes, e.g. fluidised-bed, the two reactions, methanation and WGS, can be carried out in the same reactor, the vapour necessary for the WGS reaction being mixed with the synthetic gas or injected directly into the reactor.

At low temperatures, i.e. below 170° C., the nickel (constituting the catalyst or present in the material making up the reactor walls) is likely to react with the carbon monoxide to form nickel tetracarbonyl (Ni(CO)₄), a highly toxic compound. For this reason, it is essential that all portions of the reactor are always at a temperature above 170° C. and preferably above 200° C.

The heat generated during CO conversion is approximately 2.7 kWh during the production of 1 Nm³ of methane. Controlling the temperature of the reactor, and therefore removing the heat produced by the reaction, is one of the keys to minimising the deactivation of the catalyst (sintering, etc.) and maximising the methane conversion. If the temperature of the reactor rises, the methane production falls significantly. If the temperature falls below 250° C., the methanation reaction is inhibited, since the kinetics become very slow.

The composition of the raw SNG on output from the reactor is closely related to the operating conditions of the reactor (pressure, temperature, adiabatic or isothermal nature, stoichiometry, catalyst, etc.) that govern the chemical kinetics and balances of the reactions R1, R2 and R3. These reactions generally form water, and consequently it needs to be separated. With regard to the other species (CO, CO₂ and H₂), their respective concentrations depend on the operating mode of the reactor (adiabatic or isothermal) and secondly on the temperature and/or pressure. From a thermodynamic perspective, a high pressure and a low temperature will considerably reduce the concentrations of CO and H₂. Below 250° C., the methanation reaction can be significantly inhibited. When the operation is carried out in an “adiabatic” reactor, a series of steps is also necessary to achieve an equivalent conversion quality to the isothermal reactor. In any event, the composition of the gas produced is generally incompatible with regard to the specifications for injection into natural gas networks, and upgrading steps are most often necessary to remove the water, CO₂ and/or the residual H₂. Therefore, the operating process forms a block to simplifying the chain of methods.

There are several possible technological approaches for the thermal and reaction control of an SNG production system:

Approach No. 1: Reactor Limited by the Kinetics

In the case of the adiabatic fixed-bed reactor (i.e. with no internal cooling), the reaction heat results in a rise in the temperature of the reaction medium along the reactor as the conversion progresses. By limiting the size of the equipment, the conversion is also limited and the reaction mixture leaves the reactor before reaching equilibrium. The temperature is therefore maintained below the usual limits for the catalysts. After cooling, the mixture is then injected into a second reactor, etc. An industrial process based on this principle therefore takes the form of a series of reactors with intermediate cooling between each stage until a conversion is achieved that meets requirements.

The main disadvantages of this solution are:
a multi-stage operation of reactors and exchangers (impact on the capital cost and dimensions);
an operation at high pressure (impact on the operating cost);
a risk of the premature deterioration of the catalyst by sintering (temperature peaks).

Approach No. 2: Balanced Reactor

When the quantity of catalyst present in the reactor is sufficient, the reaction is limited by the thermodynamic equilibrium. The temperature induced can, however, exceed the maximum acceptable temperature for the catalyst and lead to its deactivation by sintering of the active metals.

Diluting the reaction mixture with a gas such as water vapour, CO₂, or a thermal ballast allows the temperature to be limited. One method consists, for example, of recycling humid gas, cooled to around 250° C., from the first reactor, towards its inlet. In practice, the industrial processes utilising balanced reactors are formed of an arrangement of several reactors with recycling of a portion of the gas for some of them.

This type of methanation system often requires a prior adjustment of the H₂/CO ratio to 3 by WGS upstream to prevent, for example, coke deposits. Through 3 or 4 stages of conversion at high pressure (often higher than 20 bar), the injection specifications can be met after upgrading.

Approach No. 3: Wall-Cooled Reactor

Removing the reaction heat by the walls of the reactor, themselves cooled by a cooling fluid, is a conventional technique for controlling the temperature of the reactors in the case of exothermic reactions.

In cases of high exothermicity, the exchange surfaces required are sometimes very large. In the case of a cooled fixed-bed reactor, in order to maximise the exchange surfaces/volume ratio, the reactor generally takes the form of a multitubular reactor, the catalyst being arranged inside tubes, referred to as “TWR” (for “Throughwall Cooled Reactor”). The cooling liquid can be water, an organic liquid or a mixture of organic liquids, or a gas (N₂, CO₂, etc.). Control of the output temperature is easy and can, for example, be performed by boiling the cooling liquid (U.S. Pat. Nos. 2,662,911, 2,740,803). According to one variant, the catalyst is directly impregnated on the walls of the cooled tubes to maximise the thermal exchanges.

Another form of wall-cooled reactor consists not of arranging the catalyst in the tubes but rather of integrating a dense bundle of cooled tubes within a catalyst bed (U.S. Pat. Nos. 4,636,365, 6,958,153, 4,339,413).

Even if overall the reactor can be considered to be isothermal because of limited thermal transfers, the risks of forming hot points within the catalytic layer are, however, known to the person skilled in the art.

Similar to the technology of the balanced reactor or of the temperature-limited reactor, a prior WGS step is generally required in this type of technology to prevent the deactivation of the catalyst by coke deposit.

During the methanation of a gasification syngas, a significant pressure (P>20 bar) is necessary to dispense with the H₂ separation step (also known as “polishing”).

Approach No. 4: The “Boiling Water Reactor” (Known as “BWR”).

The BWR concept, coming from methanol production, recently adapted for the methanation of CO₂ is probably applicable to the methanation of a gasification syngas with pre-WGS. It is based on a wall-cooled double-pass tubular reactor. In this reactor, several tubes containing the catalyst are dedicated to a first pass allowing the synthetic gas to be converted into methane. On direct output from this pass, one portion of the SNG is compressed again before being mixed with the stream of feed syngas. The other portion of the first pass SNG is cooled to condense the water formed by the reactions. Methanation is then achieved in a second pass through other tubes arranged in the same reactor. The main advantage of providing a second pass is to keep a relatively constant SNG quality even if the first pass catalyst is progressively deteriorated by movement of the reaction front.

Approach No. 5: Fluidised-Bed Reactor

The utilisation of a fluidised-bed reactor is a simple and effective solution for limiting the reaction temperature. Fluidisation of the catalyst by the reaction mixture enables homogenisation of the temperatures and therefore the isothermality of the catalytic layer. The heat produced by the reaction is removed by exchangers immersed in the fluidised bed with high thermal transfer coefficients of 400 to 600 W/K·m².

With regard to the reaction, and unlike the technologies described above, methanation of the syngas in a fluidised bed does not systematically require pre-WGS. Co-injecting vapour with the syngas means the R2 (CO methanation) and R1 (WGS) reactions can be carried out in the same device.

The solutions currently proposed for this technological family are not differentiated from each other by conversion efficiency, but mainly by the methodology utilised to cool the reactor.

For example, the COMFLUX methanation process for producing SNG from syngas obtained from a carbon gasification reactor. It is based on using a fluidised bed in which are positioned vertical exchanger tubes suspended from the crown of the disengagement area (U.S. Pat. No. 4,539,016). The cooling is performed by boiling a liquid, which can be water.

PSI's methanation fluidised bed (EP1568674A1, WO2009/007061A1) is also known. This invention utilises a cooling system formed, in a similar way to the COMFLUX device, of a bundle of tubes arranged in the bed. PSI's patents claim a method for the production of SNG from biomass gasification. This method claims a solution for fluidised-bed methanation without prior treatment of the syngas on adsorption beds formed of activated carbon.

ENGIE's fluidised-bed methanation reactors are also known. These technologies essentially propose technical solutions controlling the isothermality of the reactor (by superheated vapour or by injecting liquid water into the reactor, for example).

Methods of methanation and methanolisation, i.e. hydrogenation for producing methanol, developed by ENGIE are also known, having as purpose the recovery of a stream obtained from electrolysis or co-electrolysis of water.

Lastly, methods for producing synthetic gas developed by ENGIE are also known, such as French patent applications no. 1650494, no. 1650498 and no. 1650497, in which one portion of the wholly or partly dehydrated products is recirculated for cooling the methanation reaction and also adjusting the thermodynamic equilibrium occurring in a reactor.

General Information about the Water-Gas Shift Reaction

The WGS reaction (formula 2 above) is reversible and weakly exothermic, and consists of converting CO and H₂O into H₂ and CO₂:

CO+H₂O⇄H₂+CO₂ ΔG298K=−41 kJ/mol [Formula 2]

Even though the thermodynamic equilibrium is boosted by the low temperatures, the kinetics of this reaction is nevertheless limited under these conditions if the catalyst is not suitable.

Therefore, high temperatures (350-600° C.) can be utilised to speed up the kinetics of this reaction, whereas low temperatures (190-250° C.) promote the production of hydrogen, but result in slower reaction kinetics if the choice of catalyst is not suitable. As the number of moles is constant during the reaction, the pressure has no role in the thermodynamic equilibrium of this reaction. Whereas a sur-stoichiometric water presence promotes the reaction.

On an industrial scale, most of the solutions utilise a series of adiabatic catalytic reactors operating in descending order of temperature. Beyond the benefit for the conversion, this series of reactors also makes it possible to limit the increasing temperature of the catalyst linked to the exothermicity of the reaction. As with adiabatic methanation, a heat exchanger is placed between each reactor to cool the gas mixture before injection into the next reactor. Generally, the WGS catalysts are based on iron, chromium, copper or zinc and are utilised between 200° C. and 450° C., under a pressure of 1 bar to 35 bar. Chromium makes it possible to limit the sintering of the catalyst, although replacement every 2-5 years is necessary. Cerium-based catalysts also exhibit performance levels of interest for high-temperature WGS conversion. Low-temperature WGS catalysts are mainly comprised of copper/zinc deposited on an aluminium oxide.

Some known methods, such as those described in the patent application WO 2019/234208, envisage a series of adiabatic reactors. The syngas enters the WGS catalytic reactor. On output, the gas is cooled and split into two streams, each feeding a WGS catalytic reactor of lower temperatures.

In Johnson Matthey's patent (US 2014/0264178), a syngas containing at least one sulphur compound and vapour enters into a reactor/exchanger and passes into a distributor and then into vertical tubes immersed in a fixed bed of catalysts (Co/Mo sulphide) promoting the WGS reaction. Syngas circulating co-currently outside the tubes is converted into hydrogen by the WGS reaction on contact with the catalyst. In the case of a syngas with a low H₂/CO ratio, vapour produced by a boiler is added to the syngas. The streams of syngas in the tubes and outside the tubes circulate counter-currently, unlike in the previous case.

In a 2018 patent (GB2556665), Linde proposes a method making it possible to produce hydrogen from biomass gasification. The biomass is gasified with air, at atmospheric pressure and up to 600° C., the syngas is cooled and then introduced into a WGS reactor, the products of this reaction are cooled then introduced into an electrochemical separation and compression device (7-14 bar) making it possible to separate the hydrogen output at 150-350 bar.

A patent application filed in 2009 by Haldor Topsoe (U.S. Pat. No. 7,618,558) describes a scrubbing chain for syngas produced by gasification.

A patent application filed in 2017 by Haldor Topsoe (WO 2017/186526) enables the hydrogen enrichment of a syngas comprised at least 25%, 40% or 70% on a dry basis of CO and H₂.

Membrane reactors are especially effective for the WGS reaction. The membranes integrated into the reactor makes it possible to continually extract the hydrogen produced by the reaction, thereby moving the equilibrium towards the conversion of CO into hydrogen. In this way, very high conversion rates can be achieved. Even if it is very efficient for the production of hydrogen, due to its operating principle, this reactor cannot easily enable the production of synthetic methane, because the H₂ of the syngas or produced by WGS would be continually separated as it is formed. An example of this type of method applying biomass gasification is given in patent US201783721 of the National University of Singapore.

There are numerous different technological solutions generally dedicated to the production of methane or to the production of hydrogen. However, none of the solutions mentioned above address the following technical problems:
adapting its production (biomethane or bio-hydrogen) according to the market needs, and therefore boost the installation of these production plants, which will provide the ability to adapt responsively;
mainly producing biomethane while occasionally producing hydrogen to feed the small hydrogen stations to be installed initially;
mainly producing hydrogen (mobility or industrial use) and occasionally producing biomethane (when industrial consumption is reduced (technical shutdown or stoppage of activity) or if the hydrogen mobility requirements fluctuate over time); and
quickly switching from methane production to hydrogen production, and vice versa.

Subject of the Invention

The present invention aims to remedy all or part of these drawbacks.

To this end, according to a first aspect, the present invention relates to a device for the hybrid production of synthetic dihydrogen and/or synthetic methane, which comprises:
an inlet for a stream of synthetic gas (known as “syngas”) comprising at least CO (for “carbon monoxide”) and preferably at least H₂,
a catalytic conversion reactor, configured to operate according to one of the two following alternative configurations:
   a first configuration in which the operating conditions of the reactor promote a Sabatier reaction, so as to produce an outlet gas comprising mainly methane, or
   a second configuration in which the operating conditions of the reactor promote a water gas shift reaction, so as to produce an outlet gas comprising mainly dihydrogen;
an outlet for a stream of synthetic dihydrogen and/or synthetic methane and
a control system comprising a means for selecting a configuration for operating the reactor and a control means according to the selected configuration, the reactor being configured to operate according to a given configuration as a function of the command emitted by the emission means.

These provisions make it possible to:
adapt the production (biomethane and/or biohydrogen) according to the market needs, and therefore boost the installation of these production plants, which will provide the ability to adapt responsively;
mainly produce biomethane while occasionally producing hydrogen to feed the small hydrogen stations to be installed initially;
mainly produce hydrogen (mobility or industrial use) and occasionally produce biomethane (when industrial consumption is reduced (technical shutdown or stoppage of activity) or if the hydrogen mobility requirements fluctuate over time); and
quickly switch from methane production to hydrogen production, and vice versa.

These provisions make it possible to produce a flexible device, able to produce either hydrogen or methane with a single installation and without changing the process chain implemented for the production of methane.

In some embodiments, the conversion reactor comprises a catalytic bed comprising two separate catalysts, a first catalyst being configured to promote a Sabatier reaction at medium temperature, preferably between 250° C. and 350° C.; and a second catalyst being configured to promote a water gas shift reaction at high temperature, preferably higher than 350° C.

In some embodiments, the conversion reactor comprises a catalytic bed comprising two separate catalysts, a first catalyst being configured to promote a Sabatier reaction at medium temperature, preferably between 250° C. and 350° C.; and a second catalyst being configured to promote a water gas shift reaction at low temperature, preferably between 200° C. and 250° C.

In some embodiments, the conversion reactor comprises a catalytic bed comprising a bifunctional catalyst, configured to promote a Sabatier reaction at medium temperature, preferably between 250° C. and 350° C., in the first configuration of the reactor; and to promote a water gas shift reaction at high temperature in the second configuration of the reactor, preferably higher than 350° C.

In some embodiments, the conversion reactor comprises a catalytic bed comprising a bifunctional catalyst, configured to promote a Sabatier reaction at medium temperature, preferably between 250° C. and 350° C., in the first configuration of the reactor; and to promote a water gas shift reaction at low temperature in the second configuration of the reactor, preferably between 200° C. and 250° C.

These embodiments make it possible to achieve a WGS reaction directly in the conversion reactor, either for cooling the reactor and balancing the H₂/CO ratio to the stoichiometry for CO methanation when this reactor is in the methane production configuration, or for producing dihydrogen by CO conversion when the reactor is in the dihydrogen production configuration.

For a WGS reaction at low temperature, between 200° C. and 250° C., and low pressure, and for a methanation reaction at medium temperature and high pressure, the methanation is almost completely inhibited and gives way almost entirely to WGS and therefore to the production of H₂.

In some embodiments, the device that is the subject of the present invention comprises, downstream from the conversion reactor, a water separator configured to supply the separated water to a discharge or a recovery system for water (e.g. steam production) or to an injector to feed the conversion reactor.

These embodiments make it possible to recycle the water exiting from the conversion reactor towards the inlet of said conversion reactor.

In some embodiments, the device that is the subject of the present invention comprises a means for compressing syngas to a specified pressure, the outlet pressure of the compression means being determined as a function of the command emitted by the control system.

In some embodiments, the device that is the subject of the present invention comprises a means for expanding syngas to a specified pressure, the outlet pressure of the expansion means being determined as a function of the command emitted by the control system.

These embodiments allow the inlet pressure of the conversion reactor to be adjusted to maximise the production of the product corresponding to the envisaged operating configuration of the reactor.

In some embodiments, the device that is the subject of the present invention comprises a heat exchanger immersed in the conversion reactor, said heat exchanger being configured to cool or heat the reactor to a temperature determined as a function of the command emitted by the control system.

These embodiments allow the temperature of the conversion reactor to be adjusted to maximise the production of the product corresponding to the envisaged operating configuration of the reactor.

In some embodiments, the device that is the subject of the present invention comprises a recirculator for recirculating at least part of the outlet gas towards the inlet for syngas, a quantity of recirculated gas being determined as a function of the command emitted by the control system.

These embodiments make it possible to recycle products from the conversion reactor to increase the yield of the device.

In some embodiments, the device that is the subject of the present invention comprises, downstream from the conversion reactor:
a methane output selector connected to a recirculator for recirculating methane towards the inlet for syngas, and to a methane outlet;
a dihydrogen output selector connected to a recirculator for recirculating dihydrogen towards the inlet for syngas, and to a dihydrogen outlet,
device wherein:
   when the command emitted corresponds to a configuration of the reactor to promote a water gas shift reaction, the dihydrogen output selector is configured to direct the dihydrogen towards the dihydrogen outlet and the methane output selector is configured to direct the methane towards the methane recirculator; and
   when the command emitted corresponds to a configuration of the reactor to promote a Sabatier reaction, the dihydrogen output selector is configured to direct the dihydrogen towards the dihydrogen recirculator, and the methane output selector is configured to direct the methane towards the methane outlet.

These embodiments make it possible to achieve a selective recirculation as a function of the objectives of the selected configuration.

In some embodiments, the catalytic conversion reactor is an isothermal reactor.

In some embodiments, the catalytic conversion reactor is a fluidised-bed reactor.

In some embodiments, there is only one catalytic conversion reactor.

According to a second aspect, the present invention envisages a method for the hybrid production of synthetic dihydrogen and/or synthetic methane, which comprises:
a step of selecting a configuration for operating a conversion reactor;
a step of emitting a command representative of the selected configuration,
a step of configuring the conversion reactor as a function of the command emitted according to one of the two following configurations:
   a first configuration in which the operating conditions of the reactor promote a Sabatier reaction, so as to produce an outlet gas comprising mainly methane, or
   a second configuration in which the operating conditions of the reactor promote a water gas shift reaction, so as to produce an outlet gas comprising mainly dihydrogen;
a step of inputting a stream of synthetic gas (known as “syngas”) comprising at least CO and preferably H₂;
a step of catalytic conversion reaction according to the selected configuration; and
a step of outputting a stream of synthetic dihydrogen and/or synthetic methane and

As the aims and advantages of the method are identical to those of the device that is the subject of the present invention, they are not described here.

### BRIEF DESCRIPTION OF THE FIGURES

Other advantages, aims and particular features of the invention will become apparent from the non-limiting description that follows of at least one particular embodiment of the device and method that are the subjects of the present invention, with reference to drawings included in an appendix, wherein:

FIG. 1 represents, schematically, a particular embodiment of the device that is the subject of the present invention;

FIG. 2 represents, schematically and in the form of a logical diagram, a first particular series of steps of the method that is the subject of the present invention;

FIG. 3 represents, schematically and in the form of a logical diagram, a second particular series of steps of the method that is the subject of the present invention; and

FIG. 4 represents, schematically and in the form of a logical diagram, a third particular series of steps of the method that is the subject of the present invention.

### DESCRIPTION OF THE EMBODIMENTS

The present description is given in a non-limiting way, in which each characteristic of an embodiment can be combined with any other characteristic of any other embodiment in an advantageous way.

Note that the figures are not to scale.

It is noted that the term “synthetic methane” refers, more generally, to synthetic natural gas, which can comprise other chemical species in addition to the methane produced.

Three ranges of temperature operating conditions are defined:
the “low” temperatures are temperatures below 250° C. and above 200° C.;
the “medium” temperatures are temperatures between 250° C. and 350° C.; and
the “high” temperatures are temperatures above 350° C.

Two ranges of pressure operating conditions are also defined:
the “low” pressures are pressures strictly below a predefined pressure limit, for example, atmospheric pressure, 2 bar or 3 bar; and
the “high” pressures are pressures above the predefined pressure limit in bars.

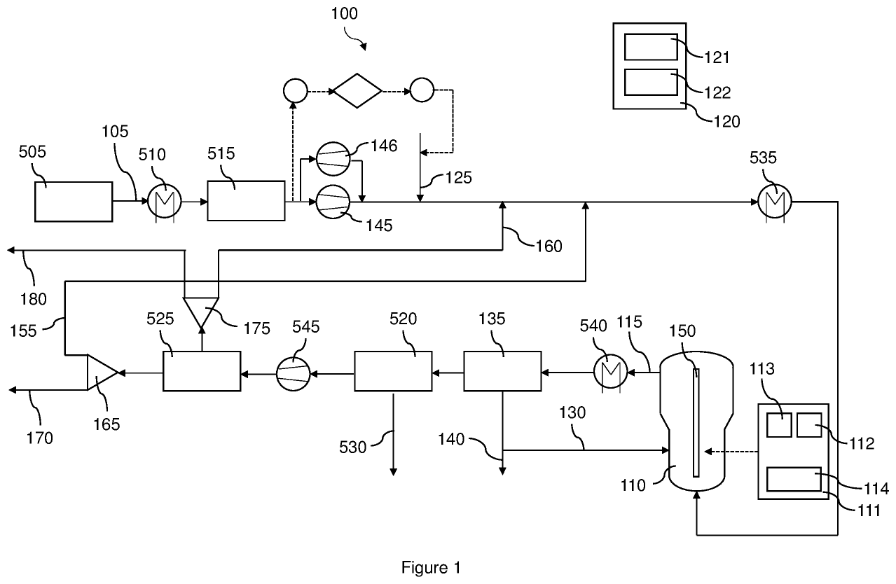

FIG. 1, which is not to scale, shows a schematic view of an embodiment of the device 100 that is the subject of the present invention. The device 100 for the hybrid production of synthetic dihydrogen and/or synthetic methane comprises:
an inlet 105 for a stream of synthetic gas, known as “syngas”, comprising at least CO and preferably at least H₂;
a catalytic conversion reactor 110, configured to operate according to one of the two following alternative configurations:
   a first configuration in which the operating conditions of the reactor promote a Sabatier reaction at medium temperature and high pressure, so as to produce an outlet gas comprising mainly methane, or
   a second configuration in which the operating conditions of the reactor promote a water gas shift reaction at high temperature, or a water gas shift reaction at low temperature, and at low pressure, so as to produce an outlet gas comprising mainly dihydrogen;
an outlet 115 for a stream of synthetic dihydrogen and/or synthetic methane; and
a control system 120 comprising a means 121 for selecting a configuration for operating the reactor and a means 122 for emitting a command representative of the selected configuration, the reactor being configured to operate according to a given configuration as a function of the command emitted by the emission means.

The inlet 105 for a stream of gas generally means any line allowing syngas to be conveyed towards a syngas inlet (unnumbered) of the conversion reactor 110. The precise nature of the inlet 105 depends on the operating conditions determined in terms of flow rate, in particular, and on the nature of the syngas to be transported.

In a particular embodiment, such as that shown in FIG. 1, the inlet 105 is fed with syngas by a gasifier 505 of waste, biomass and/or carbonaceous residue. It is noted that the term “gasifier” and “gasification reactor” are equivalent here.

Gasification corresponds to a thermal degradation of the biomass, waste or carbonaceous residue, which undergoes successively drying and then devolatilisation, or pyrolysis, of the organic matter to produce a carbonaceous residue (the “char”), a synthetic gas (known as “syngas”), and condensable compounds (tars). The carbonaceous residue can then be oxidised by the gasification agent (water vapour, air, oxygen, carbon dioxide) to produce a gas mainly composed of H₂ and CO. Depending on its nature, this gasification agent may also react with the tars or the major constituent gases. Thus, in the case of water vapour (H₂O), then a WGS (for “Water Gas Shift”) reaction also occurs in the gasification reactor 505.

The pressure of the gasification reactor 505 has little effect on this reaction. In contrast, the equilibrium is strongly linked to the temperature of the reactor and to the “initial” composition of the reagents. The syngas obtained consists of a mixture of mostly non-condensable gases (H₂, CO, CO₂, CH₄, Cₓ), condensable compounds (tars), particles (char, coke, elutriated bed material), and inorganic gases (alkalis, heavy metals, H₂S, HCl, NH₃, etc.). After the elimination of impurities, the majority gases can be transformed into many energy carriers, including biomethane and biohydrogen. For the production of these two compounds, the H₂/CO ratio in the syngas is a decisive factor. On output from the gasification reactor 505, this ratio does not generally exceed 2 but sometimes ratios higher than 6, under certain conversion conditions, can be obtained.

In a particular embodiment, such as that shown in FIG. 1, the device 100 comprises a means 510 for cooling products from the gasifier 505.

These embodiments make it possible to adapt the temperatures of the gas produced to the operation of the equipment of the device 100.

In a particular embodiment, such as that shown in FIG. 1, the device 100 comprises a means 515 for eliminating impurities from products from the gasifier 505. The elimination means 515 can be positioned upstream or downstream from the cooling means 510 if the device 100 comprises such a cooling means 510.

These embodiments make it possible to adapt the quality of the gas produced to the operation of the equipment of the device 100.

The precise nature of the elimination means 515 depends on the nature of the impurities to be eliminated. Such elimination means 515 are well known to the person skilled in the art. For example, such an elimination means 515 is a scrubber. Such a scrubber can utilise wet neutralisation, dry adsorption or neutralisation, depending on the use determined.

In some embodiments, the device 100 comprises a plurality of elimination means 515 in cascade incorporating a multitude of unitary operations or processes arranged in series or in parallel (absorption, physical and/or chemical adsorption on, for example, activated carbon, zeolite, ash, metals, etc.). In some variants, between two impurity elimination stages, the device 100 comprises a means (not shown) for cooling syngas and/or a syngas compressor (not shown).

In some embodiments, the device 100 comprises a means (not shown) for removing dust from the syngas. Such a dust removal means is, for example, is a venturi, multicyclone or filter type.

In a particular embodiment, such as that shown in FIG. 1, the device 100 comprises a means 145 for compressing syngas to a specified pressure, the outlet pressure of the compression means 145 being determined as a function of the command emitted by the control system 120.

This compression means 145 is, for example, a centrifugal, axial, vane, screw, lobe, piston or scroll type of compressor. This compression means 145 is configured, for example, to bring the syngas to a pressure between 1 and 80 bar, and preferably between 1 and 15 bar.

In a particular embodiment, not shown in FIG. 1, the device 100 comprises a means 146 for expanding syngas to a specified pressure, the outlet pressure of the expansion means being determined as a function of the command emitted by the control system 120.

This expansion means 146 can be any type known to the person skilled in the art and adapted to the use considered. In a particular embodiment, such as that shown in FIG. 1, the device 100 comprises, upstream from the inlet for syngas into the conversion reactor 110, a heat exchanger 535. This heat exchanger, which can be a plate or tube and shell exchanger or a series of heat exchangers (not shown), for example, is configured to heat or cool the syngas to a temperature compatible with the specific configuration of the conversion reactor 110.

Preferably, this exchanger 535 can make it possible to ensure a minimum inlet temperature for the reactor between 170° C. and 230° C., and preferably above 170° C. to prevent the formation of nickel tetracarbonyl (if a nickel-based reactor or catalyst) which is a poison in the gas produced.

The catalytic conversion reactor 110 is, preferably, an isothermal reactor. Preferably, this conversion reactor 110 is a wall-cooled isothermal reactor exchanger or a cascade of isothermal reactors. More preferably, this conversion reactor 110 is a fluidised-bed isothermal reactor. Preferably, this reactor 110 is a single fluidised-bed isothermal reactor. More preferably, this conversion reactor 110 is a dense or bubbling fluidised-bed isothermal reactor. The term “dense fluidised-bed isothermal reactor” refers to a reactor configured to operate according to a temperature between 200° C. and 600° C. and according to a pressure between 1 and 80 bar.

This reactor 110 is configured to operate according to two thermodynamic equilibrium configurations, or systems:
a first configuration in which the operating conditions of the reactor promote a Sabatier reaction at medium temperature and high pressure, so as to produce an outlet gas comprising mainly methane, or
a second configuration in which the operating conditions of the reactor promote a water gas shift reaction at high temperature, or a water gas shift reaction at low temperature, and at low pressure, so as to produce an outlet gas comprising mainly dihydrogen.

The choice of the temperature range of this second configuration will be a function of the catalytic bed 111 introduced into 110 to ensure the water-gas or WGS reaction. This configuration requires significant operating ranges in terms of pressure and temperature in particular.

It is noted that the invention is not limited to a single reactor, and this can implement a plurality of reactors, of identical or different types, in parallel or in series to obtain the reaction product envisaged.

To be able to achieve the two reactions according to the two configurations in question, the reactor 110 utilises a catalytic bed 111. Such a catalytic bed 111 can be formed of:
either a mixture of two separate catalysts, 112 and 113, each configured to promote one of the two configurations, wherein each catalyst can utilise different metals for example;
or a single bifunctional catalyst 114 configured to promote, based on other reaction parameters (temperature or pressure for example), one or other of the configurations.

In the case of separate catalysts:
the catalyst promoting the Sabatier reaction can be based on Ni/Al₂O₃, Ni/Pr/Al₂O₃, Ruthenium, for example; and
the catalyst promoting the WGS reaction can be based, for example, on CuO/ZnO/Al₂O₃, ZnO, Cr₂O₃, KOH/Pt/Al₂O₃, Pt-CeOx/Al₂O₃, CuO/Al₂O₃, for conversions at low temperature, for example around 200° C., or Fe₂O₃/Cr₂O₃, Au—Fe₂O₃, Au—CeO₂, Au—TiO₂, Ru—ZrO₂, Rh—CeO₂, Pt—CeO₂ or Pd—CeO₂, for conversions at high temperature, for example around 450° C.

In the first configuration, the objective of the reactor 110 is to maximise the production of synthetic methane (CH₄). The syngas can be converted into biomethane by the catalytic methanation reaction of the CO, also called the “Sabatier reaction”. This reaction, which has rapid kinetics at the temperatures utilised, are characterised by very high exothermicity.

To maximise the production of CH₄, the H₂ and CO should have a stoichiometric ratio of about 3:1. This ratio can be obtained by performing an additional WGS reaction positioned upstream from 110.

In some variants (not shown), the device 100 comprises a dedicated WGS reactor comprising specific catalysts. Such a specific catalyst is, for example, based on Cu—Zn—Al₂O₃, Fe₂O₃/Cr₂O₃. In other preferred variants, such as that shown in FIG. 1, the additional WGS reaction is performed directly in the same conversion reactor 110.

Regardless of the variant chosen, a supply of water (vapour or liquid) is therefore necessary.

In some particular embodiments, such as that shown in FIG. 1, the device 100 comprises an injector 125 injecting vapour into the stream of syngas and/or an injector 130 injecting liquid water or vapour into the catalytic reactor, a quantity of water and/or vapour injected by at least one injector being realised as a function of the command emitted by the control system 120. The injector 125 can be positioned upstream or downstream from a possible recirculation, 155 or 160, described below.

The injector 125 for injecting vapour is, for example, a branch in the inlet line 105 associated to a production means (not shown) to bring the water to a temperature corresponding to the vapour state at the operating conditions of the inlet 105 for syngas.

The injector 130 for injecting water into the conversion reactor 110 is, for example, a branch feeding liquid water or vapour into the conversion reactor 110. This branch can be fed with external water or with recycled water in the device 100.

In some particular embodiments, such as that shown in FIG. 1, the device 100 comprises, downstream from the conversion reactor 110, a water separator 135 configured to supply the separated water to a water discharge 140 or to an injector, 125 and/or 130, after its vapour phase transformation, for example through a heat exchanger (not shown). The separator 135 can be of condenser type, for example. The water separator 135 is configured to dehydrate the stream produced on output from the conversion reactor 110, by cooling, for example, to a temperature corresponding to a temperature below or equal to the dew point temperature of the water at the operating conditions of the device 100.

In some particular embodiments, such as that shown in FIG. 1, the device 100 comprises, upstream from the water separator 135, at least one heat exchanger 540. At least one heat exchanger 540, of type plate or tube and shell exchanger for example, is configured to cool the products from the conversion reactor 110 to a temperature corresponding to a temperature above or equal to the dew point temperature of the water at the operating conditions of the device 100.

Inside the conversion reactor 110, the CO₂ also present in the syngas can also produce CH₄ by methanation reaction of the CO₂ if the hydrogen is in sur-stoichiometry for CO methanation.

In the second configuration, the objective of the reactor 110, or of the plurality of reactors 110, is to maximise the production of synthetic hydrogen. To this end, the WGS reaction can be specifically implemented in the single conversion reactor 110 or in a plurality of conversion reactors 110. For this, a greater quantity of water is injected than the quantity mentioned in the case of methanation, so as to maximise the production of hydrogen.

Regardless of the configuration, the products from the conversion reactor 110 comprise water in excess or in products, carbon dioxide, hydrogen and methane in proportions that vary according to the configuration implemented.

The outlet 115 for a stream of synthetic dihydrogen and/or synthetic methane means any line allowing the products from the conversion reactor 110 to be carried away from the conversion reactor 110.

The products passing through the outlet 115 are preferably adjusted to the specifications of use downstream from the conversion reactor 110 as described below.

These specifications correspond, for example, in the case of synthetic methane that can be injected into the natural gas networks, to:
a higher heating value between 9.5 and 12.8 kWh/Nm³;
a Wobbe index between 12.01 and 15.70 kWh/Nm³;
a relative density between 0.555 and 0.7;
a CO₂ content of less than 2.5%; and
a dihydrogen content of less than 6%, or less than 2%, depending on the use.

In some particular embodiments, such as that shown in FIG. 1, the device 100 comprises a separator 520 for separating carbon dioxide from the stream on output from the conversion reactor 110.

This separator 520 is, for example, a device configured to perform the adsorption (physical or chemical) or pressure swing adsorption, membrane permeation or cryogenics of the carbon dioxide of the stream, and direct it towards a discharge or a recovery system 530 for carbon dioxide.

In some particular embodiments, such as that shown in FIG. 1, the device 100 comprises at least one recirculator, 155 and/or 160, for recirculating at least part of the outlet gas towards the inlet 105 for syngas, a quantity of recirculated gas being determined as a function of the command emitted by the control system 120.

The term “recirculator”, 155 and 160, refers to a line transporting a stream of gas towards the inlet 105 for syngas. This stream of gas can be a stream of hydrogen 160 or synthetic methane 155, as a function of the command emitted by the control system 120. For example, if the control system 120 has configured the device 100 for producing hydrogen, the residual methane is recirculated by the “recirculator” 155, whereas if the control system 120 has configured the device 100 for producing methane, it is the dihydrogen which is recirculated by the “recirculator” 160. Alternatively, the product whose production is maximised by the configuration of the device 100 can also be recirculated so as to keep the flow rate passing through the conversion reactor 110 constant.

In some particular embodiments, such as that shown in FIG. 1, the device 100 comprises, downstream from the conversion reactor 110:
a methane output selector 165 connected to a recirculator 155 for recirculating methane towards the inlet 105 for syngas, and to a methane outlet 170;
a dihydrogen output selector 175 connected to a recirculator 160 for recirculating dihydrogen towards the inlet 105 for syngas, and to a dihydrogen outlet 180,
device wherein:
   when the command emitted corresponds to a configuration of the reactor to promote a water gas shift reaction, the dihydrogen output selector is configured to direct the dihydrogen mainly towards the dihydrogen outlet and the methane output selector is configured to direct the methane mainly towards the methane recirculator; and
   when the command emitted corresponds to a configuration of the reactor to promote a Sabatier reaction, the dihydrogen output selector is configured to direct the dihydrogen mainly towards the dihydrogen recirculator, and the methane output selector is configured to mainly direct the methane towards the methane outlet.

For clarity, “mainly” means a proportion higher than 50%.

Preferably, the amount of dihydrogen and/or the amount of methane recirculated are adjusted to produce a mixture of dihydrogen and methane in the proportions given.

The recirculation of residual methane and residual hydrogen, 155 and 160, can inject the residual gas downstream from the compression means 145, for example during the use of a membrane separation in 525. The H₂ is thus obtained in the permeate, therefore at low pressure. In the second configuration, H₂ is taken up by a recovery system 180 downstream. In the case of the first configuration, the low-pressure permeate H₂ is fed upstream from 145 at a lower pressure than the operating pressure of 110.

In some particular embodiments, such as that shown in FIG. 1, the device 100 comprises, upstream from the dihydrogen output selector 175, a dihydrogen separator 525.

Such a dihydrogen separator 525 is, for example, a device for performing a membrane permeation, pressure swing adsorption and/or and electrical compressor and/or electrochemical compression.

On output from this hydrogen separation:
in the case of biomethane production: the small quantity of hydrogen present in the gas on output from the syngas conversion reactor is mainly separated from the biomethane, which can therefore be used in the transportation or distribution grids, or in a mobility station; all or part of the small quantity of hydrogen separated can be recirculated towards the stream 105 feeding the syngas catalytic conversion reactor 110; and
in the case of biohydrogen production: the large quantity of hydrogen present in the gas on output from the syngas conversion reactor is separated from the rest of the gas, thus producing a biohydrogen with sufficient purity to be used in an industrial network or in a mobility station; the rest of the gas can be recirculated in full or in part towards the stream 105 feeding the syngas catalytic conversion reactor 110.

In a particular embodiment, such as that shown in FIG. 1, the device 100 comprises a means 545 for compressing products from the conversion reactor 110 to a specified pressure, this pressure corresponding to a nominal pressure of use for said products or to an operating pressure of the conversion reactor 110 with a view to the recirculation of a portion of the reaction products.

This compression means 545 is, for example, a centrifugal, axial, vane, screw, lobe or scroll type of compressor. On output from this compression means, the reaction products preferably have a pressure of between 4 and 80 bar.

The means, 135, 520 and 525, 545, can be reversed.

In some particular embodiments, such as that shown in FIG. 1, the device 100 comprises a heat exchanger 150 immersed in the conversion reactor, said heat exchanger being configured to cool or heat the reactor 110 to a temperature determined as a function of the command emitted by the control system 120.

Such a heat exchanger 150 is, for example, formed of horizontal, vertical or inclined tubes, or a plate heat exchanger or wall-cooling of the reactor 110 or of the multiplicity of reactors 110.

The control system 120 is, for example, an electronic calculation circuit configured to:
receive a manual or automatic configuration selection via the selection means 121;

and
emit a configuration command via the emission means 122.

The selection means 121 is, for example, a mechanical, electrical or electronic interface allowing a configuration to be selected from the two configurations available.

The emission means 122 is, for example, an electronic control circuit, configured to adapt operating variables of the device 100 to correspond to the configurations available.

These operating variables are at least one of the following:
the pressure of the conversion reactor 110 adjusted by the means 145 or 146 and/or by a pressure regulating valve (not shown) positioned downstream from 110: the pressure is a variable that considerably boosts the methanation reaction. Thus, for the production of biomethane the pressure of the reactor is high, preferably higher than atmospheric pressure and even more preferably higher than 3 bar, while it is lower (preferably less than 3 bar, and even more preferably less than 2 bar, and even more preferably close to atmospheric pressure) for the production of biohydrogen by Water-Gas Shift;
the temperature of the conversion reactor 110 adjusted by the systems 150 and/or 535 or the injection of water 130: the two reactions (methanation and Water-Gas Shift) are exothermic, thus boosted by the low temperatures. However, the Water-Gas Shift catalyst in the reactor 110 is active at a low or high temperature. Therefore, the temperature of the reactor is preferably between 250° C. and 350° C. for the production of biomethane, and preferably between 200° C. and 250° C., or above or equal to 350° C., for the production of biohydrogen based on the WGS catalytic function contained in the catalyst bed 111;
the flow rate of the vapour added to the syngas or the water vapour content of the syngas: the vapour flow rate affects the thermodynamic equilibrium, and therefore the production of biomethane or biohydrogen. For the production of biomethane, the fraction by volume of vapour in the syngas conversion reactor 110 is preferably between 0 and 30% vol and preferably between 10 and 30% vol, versus 20 to 80% vol and preferably between 30 and 50% vol for the production of biohydrogen. It is noted that the fraction by volume of vapour comprises the vapour on input to the syngas conversion reactor 110 and, when the reactor 110 is cooled by injecting cooling water 130, the injected water that vaporises on contact with the hot catalyst bed 111.

This high content of water vapour added or contained in the syngas in the case of the production of biohydrogen makes it possible to discourage the methanation reaction at the expense of the water-gas reaction;
the gas fraction recirculated: in biomethane or biohydrogen production mode, the device 100 generates a residual gas. In the case of biomethane production, the hydrogen separated from the biomethane can be recycled towards the inlet 105 of the conversion reactor 110 in order to be transformed into biomethane by methanation or recovered as a small-scale biohydrogen production. In the case of biohydrogen production, the residual gas (comprised mainly of CH₄ and CO) can be recycled towards the inlet 105 of the syngas conversion reactor 110 in order to increase the biohydrogen yield. The gas fractions recirculated 155 and/or 160 towards the inlet 105 of the syngas conversion reactor 110 can vary from 0 to 100% according to the operating modes.

To maximise the production of hydrogen, specific operating conditions must be implemented:
an increase in the temperature equal to or above 350° C. promotes the production of biohydrogen if the catalyst bed 111 contains a WGS catalyst having a high-temperature WGS catalytic function;
a decrease in the temperature equal to or below 250° C. does not allow the methanation reaction to take place and promotes the production of biohydrogen if the catalyst bed 111 contains a WGS catalyst having a low-temperature WGS catalytic function;
the production of biohydrogen increases significantly when the vapour content of the syngas rises;
a decrease in the pressure of the catalytic conversion reactor 110 reduces the production of biomethane and boosts the production of biohydrogen;
the production of biohydrogen increases when the recirculation of the residual methane increases.

Based on the effects described above, and taking into account the energy impact of the settings for the different parameters mentioned above, the “nominal” conditions of the process for high-temperature Water-Gas Shift biohydrogen production can be, for example, the following:

TABLE 1

| | | |
|---|---|---|
| Pressure of the reactor 110 | 1.2 | bar |
| Temperature of the reactor 110 | 450 | ° C. |
| Vapour content of the syngas | 50 | % mol |
| Rate of Recirculation 165 | 80 | % |
| Rate of Recirculation 175 | 0 | % |

Note that the rate of recirculation 165 corresponds to the ratio between the stream in the recirculator 155 and the sum of the streams in the recirculator 155 and on output 170. Note that the rate of recirculation 175 corresponds to the ratio between the stream in the recirculator 160 and the sum of the streams in the recirculator 160 and on output 180.

Under these operating conditions, the composition of the various key streams of the process is as follows:

TABLE 2

| | Stream - | Stream - | Stream - | Stream - |
|---|---|---|---|---|
| | Methanation | Catalytic | Biohydrogen | Residual |
| | reactor | reactor | recoverable | gas output |
| Parameters | input | output | 170 | process 180 |
| Pressure | 1.40 | 1.20 | 10.00 | 10.00 |
| (BarA) | | | | |
| Temperature | 400.00 | 450.00 | 240.80 | 240.80 |
| (° C.) | | | | |
| Molar flow rate | 20.64 | 19.97 | 3.26 | — |
| (kmol/h) | | | | |
| CO₂ content | 10.13% | 17.43% | 0.00% | 0.00% |
| (% mol) | | | | |
| H₂O content | 36.87% | 32.23% | 0.00% | 0.00% |
| (% mol) | | | | |
| CO content | 9.00% | 1.24% | 0.00% | 0.00% |
| (% mol) | | | | |
| CH₄ content | 27.63% | 31.91% | 0.00% | 0.00% |
| (% mol) | | | | |
| H₂ content | 15.28% | 17.19% | >99.99% | 0.00% |
| (% mol) | | | | |
| C₂H₄ content | 1.10% | 0.00% | 0.00% | 0.00% |
| (% mol) | | | | |

Based on the effects described above, and taking into account the energy impact of the settings for the different parameters mentioned above, the “nominal” conditions of the process for low-temperature Water-Gas Shift biohydrogen production can be, for example, the following:

TABLE 3

| | | |
|---|---|---|
| Pressure of the reactor | 1.2 | bar |
| Temperature of the reactor | 200 | ° C. |
| Vapour content of the syngas | 50 | % mol |
| Rate of Recirculation 165 | 90 | % |
| Rate of Recirculation 175 | 0 | % |

Under these operating conditions, the composition of the various key streams of the process is as follows:

TABLE 4

| | Stream - | Stream - | Stream - | Stream - |
|---|---|---|---|---|
| | Methanation | Catalytic | Biohydrogen | Residual |
| | reactor | reactor | recoverable | gas output |
| Parameters | input | output | 170 | process 180 |
| Pressure | 1.30 | 1.10 | 10.00 | 10.00 |
| (BarA) | | | | |
| Temperature | 200.00 | 200.00 | 250.45 | 250.45 |
| (° C.) | | | | |
| Molar flow rate | 22.12 | 22.41 | 5.22 | — |
| (kmol/h) | | | | |
| CO₂ content | 9.52% | 17.65% | 0.00% | 0.00% |
| (% mol) | | | | |
| H₂O content | 34.57% | 25.03% | 0.00% | 0.00% |
| (% mol) | | | | |
| CO content | 7.56% | 0.07% | 0.00% | 0.00% |
| (% mol) | | | | |
| CH₄ content | 27.26% | 26.91% | 0.00% | 0.00% |
| (% mol) | | | | |
| H₂ content | 14.75% | 24.51% | >99.99% | 0.00% |
| (% mol) | | | | |
| C₂H₄ content | 6.33% | 5.83% | 0.00% | 0.00% |
| (% mol) | | | | |

With regard to biomethane:
an average temperature of between 250° C. and 350° C. promotes the production of biomethane—preferably a reaction below 350° C., and preferably below 320° C. and more preferably below 300° C., and preferably above or equal to 250° C. is implemented in methanation mode in order to limit the production of biohydrogen; below 250° C. the methanation reaction is limited by the kinetics, because the reactions are unable to start or are too slow;
as with the temperature, a lower water vapour content in the syngas feeding the catalytic conversion reactor promotes the production of biomethane;
a higher pressure of the catalytic conversion reactor promotes the production of biomethane in the process chain proposed;
the recirculation of the hydrogen stream towards the syngas feeding the catalytic conversion reactor 110 has no significant impact on the production of biomethane—this is because the quantity of residual hydrogen present in the stream on output from the catalytic conversion reactor in methanation mode is very low due to its consumption by the methanation reaction.

Based on the effects described above, and taking into account the energy impact of the settings for the different parameters mentioned above, the “nominal” conditions of the process for producing biomethane by methanation are, for example, the following:

TABLE 5

| | | |
|---|---|---|
| Pressure of the reactor | 4.7 | bar |
| Temperature of the reactor | 300 | ° C. |
| Vapour content of the syngas | 20 | % mol |
| Rate of Recirculation 165 | 0 | % |
| Rate of Recirculation 175 | 100 | % |

Under these operating conditions, the composition of the various key streams of the process is as follows:

TABLE 6

| | Stream - | Stream - | Stream - | Stream - |
|---|---|---|---|---|
| | Methanation | Catalytic | Biohydrogen | Residual |
| | reactor | reactor | recoverable | gas output |
| Parameters | input | output | 170 | process 180 |
| Pressure | 4.90 | 4.70 | 10.00 | 10.00 |
| (BarA) | | | | |
| Temperature | 250.00 | 300.00 | 77.78 | 77.78 |
| (° C.) | | | | |
| Molar flow rate | 9.49 | 7.16 | — | 1.66 |
| (kmol/h) | | | | |
| CO₂ content | 21.45% | 36.86% | 0.00% | 2.43% |
| (% mol) | | | | |
| H₂O content | 19.86% | 32.64% | 0.00% | 0.23% |
| (% mol) | | | | |
| CO content | 17.47% | 0.03% | 0.00% | 0.09% |
| (% mol) | | | | |
| CH₄ content | 6.35% | 29.47% | 0.00% | 97.08% |
| (% mol) | | | | |
| H₂ content | 32.49% | 1.00% | 0.00% | 0.16% |
| (% mol) | | | | |
| C₂H₄ content | 2.38% | 0.00% | 0.00% | 0.00% |
| (% mol) | | | | |

As can be understood, the aim of the present invention is to convert the syngas, for example from biomass/waste/residue, into biomethane or biohydrogen in a flexible way by simply modifying certain operating conditions while keeping the same equipment, the same process chain and the same catalyst bed 111. A hybrid fluidised-bed catalytic conversion reactor for syngas utilising a mixture of catalysts, a single low-yield catalyst or a bifunctional catalyst, makes it possible to carry out these conversions by operating:
at a medium temperature, between 250° C. and 350° C.:
   high pressure, preferably higher than atmospheric pressure, and preferably higher than 2 bar and more preferably higher than 3 bar, and preferably lower than 80 bar and more preferably lower than 20 bar, and
   low water vapour content, preferably between 0 and 30% vol and more preferably between 10 and 30% vol, for the production of biomethane,
at a high temperature, preferably above 350° C.:
   low pressure, preferably between 1 and 2 bar, and
   high water content, preferably between 30% vol and 80% vol, for the production of biohydrogen if the catalyst bed 111 contains a so-called “high-temperature” WGS catalyst,
or at a low temperature, preferably between 200° C. and 250° C.:
   low pressure, preferably between 1 and 2 bar, and
   high water content, preferably between 30% vol and 80% vol, for the production of biohydrogen if the catalyst bed 111 contains a so-called “low-temperature” WGS catalyst.

Alternatively, a plurality of reactors can be utilised in series or in parallel. Whereas an excess of vapour is conventionally used for limiting the methanation reaction during the conversion of syngas into biohydrogen by the Water-Gas Shift reaction, the present invention allows the methanation reaction to be limited by controlling the pressure, temperature, water vapour content, and also the methane content in the syngas conversion reactor. In effect, by recirculating more or less of the stream of residual gas rich in CH₄ towards the syngas conversion reactor in WGS mode, the thermodynamic equilibrium and the reaction kinetics driving the biomethane production are discouraged, which further limits the methanation reaction.

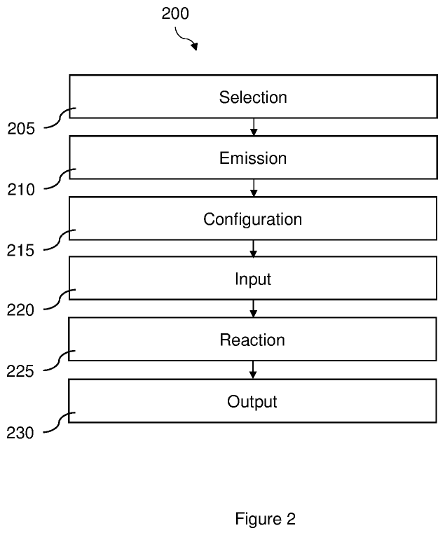

FIG. 2 shows, schematically, an embodiment of the method 200 that is the subject of the present invention. This method 200 for the hybrid production of synthetic dihydrogen and/or synthetic methane comprises:
a step 205 of selecting a configuration for operating a conversion reactor;
a step 210 of emitting a command representative of the selected configuration;
a step 215 of configuring the conversion reactor as a function of the command emitted according to one of the two following configurations:
a first configuration in which the operating conditions of the reactor promote a Sabatier reaction, so as to produce an outlet gas comprising mainly methane, or
a second configuration in which the operating conditions of the reactor promote a water gas shift reaction, so as to produce an outlet gas comprising mainly dihydrogen;
a step 220 of inputting a stream of synthetic gas, (known as “syngas”);
a step 225 of catalytic conversion reaction according to the selected configuration; and
a step 230 of outputting a stream of synthetic dihydrogen and/or synthetic methane.

Performance of the steps of:
selection 205;
   emission 210;
   inputting 220 a stream of syngas;
   reaction 225; and
   output 230
   is described with reference to FIG. 1 and in particular respectively:
   the selection means 121;
   the emission means 122;
   the inlet 105 for a stream of syngas;
   the reaction reactor 110; and
   the outlet 115 for reaction products.

The configuration step 215 is carried out with all the operational adjustments described with reference to FIG. 1 concerning the configuration for the production of synthetic dihydrogen or biomethane.

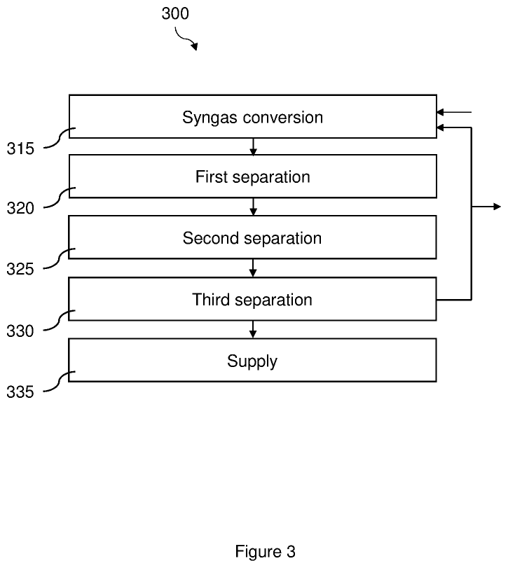

FIG. 3 shows, schematically, a particular embodiment of the method 300 that is the subject of the present invention when the method 200 is in the methane production configuration. In this embodiment, the method 300 comprises:
a step 315 of converting a stream of syngas by utilising a conversion reactor 110, which can include a step (unnumbered) of supplying water directly into the reactor 110 or into the inlet stream (unnumbered);
a first 320, second 325 and third 330 separation steps, each of these separation steps, 320, 325 and 330, being of a distinct type from amongst:
   a separation of water;
   a separation of CO₂; and
   a separation of dihydrogen;
optionally, a step (unnumbered) of recirculating residual methane on output from the third separation step 330; and
a step 335 of supplying dihydrogen for a dedicated use or storage.

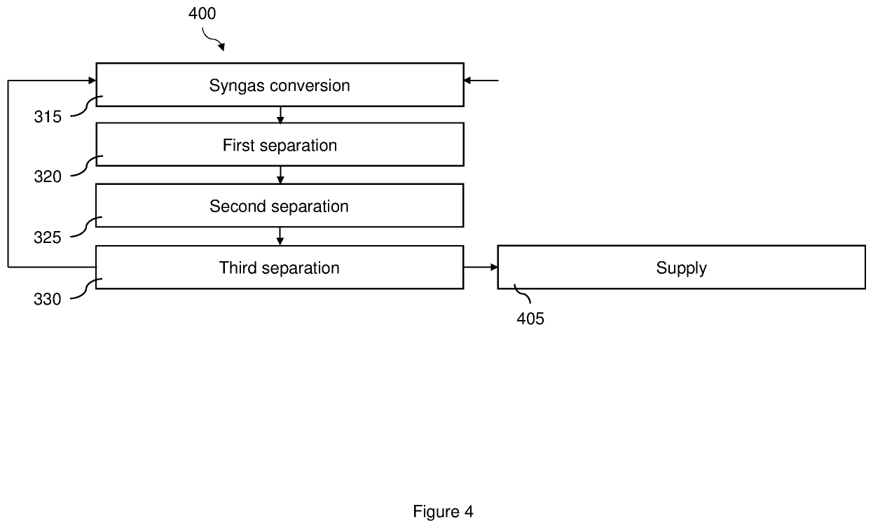

FIG. 4 shows, schematically, an embodiment of the method 400 that is the subject of the present invention when the method 200 is in the dihydrogen production configuration. In this embodiment, the method 400 comprises:
a step 315 of converting a stream of syngas by utilising a conversion reactor 110, which can include a step (unnumbered) of supplying water directly into the reactor 110 and/or into the inlet stream (unnumbered);
a first 320, second 325 and third 330 separation steps, each of these separation steps, 320, 325 and 330, being of a distinct type from amongst:
   a separation of water;
   a separation of CO₂; and
   a separation of dihydrogen;
optionally, a step (unnumbered) of recirculating residual dihydrogen on output from the third separation step 330; and
a step 405 of supplying methane for a dedicated use or storage.

1. Device for the hybrid production of synthetic dihydrogen and/or synthetic methane, comprising:
an inlet for a stream of synthetic gas (known as “syngas”), comprising at least CO;
a catalytic conversion reactor, configured to operate according to one of the two following alternative configurations:
a first configuration in which the operating conditions of the reactor promote a Sabatier reaction, so as to produce an outlet gas comprising mainly methane, or
a second configuration in which the operating conditions of the reactor promote a water gas shift reaction, so as to produce an outlet gas comprising mainly dihydrogen;
an outlet for a stream of synthetic dihydrogen and/or synthetic methane; and
a control system comprising a selector for selecting a configuration for operating the reactor and a means for emitting a command representative of the selected configuration, the reactor being configured to operate according to a given configuration as a function of the command emitted by the emission means.

2. Device according to claim 1, wherein the conversion reactor comprises a catalytic bed comprising two separate catalysts, a first catalyst being configured to promote a Sabatier reaction at medium temperature, preferably between 250° C. and 350° C.; and a second catalyst being configured to promote a water gas shift reaction at high temperature, preferably higher than 350° C.

3. Device according to claim 1, wherein the conversion reactor comprises a catalytic bed comprising two separate catalysts, a first catalyst being configured to promote a Sabatier reaction at medium temperature, preferably between 250° C. and 350° C., and a second catalyst being configured to promote a water gas shift reaction at low temperature, preferably between 200° C. and 250° C.

4. Device according to claim 1, wherein the conversion reactor comprises a catalytic bed comprising a bifunctional catalyst, configured to promote a Sabatier reaction at medium temperature, preferably between 250° C. and 350° C., in the first configuration of the reactor; and to promote a water gas shift reaction at high temperature in the second configuration of the reactor, preferably higher than 350° C.

5. Device according to claim 1, wherein the conversion reactor comprises a catalytic bed comprising a bifunctional catalyst, configured to promote a Sabatier reaction at medium temperature, preferably between 250° C. and 350° C., in the first configuration of the reactor; and to promote a water gas shift reaction at low temperature in the second configuration of the reactor, preferably between 200° C. and 250° C.

6. Device according to claim 1, which comprises an injector injecting vapour into the stream of syngas and/or an injector injecting liquid water or vapour into the catalytic reactor, a quantity of water and/or vapour injected by at least one injector being realised as a function of the command emitted by the control system.

7. Device according to claim 6, which comprises, downstream from the conversion reactor, a water separator configured to supply the separated water to a water discharge or to an injector.

8. Device according to claim 1, which comprises a means for compressing syngas to a specified pressure, the outlet pressure of the compression means being determined as a function of the command emitted by the control system.

9. Device according to claim 1, which comprises a heat exchanger immersed in the conversion reactor, said heat exchanger being configured to cool or heat the reactor to a temperature determined as a function of the command emitted by the control system.

10. Device according to claim 1, which comprises a recirculator for recirculating at least part of the outlet gas towards the inlet for syngas, a quantity of recirculated gas being determined as a function of the command emitted by the control system.

11. Device according to claim 10 which comprises, downstream from the conversion reactor:
a methane output selector connected to a recirculator for recirculating methane towards the inlet for syngas, and to a methane outlet;
a dihydrogen output selector connected to a recirculator for recirculating dihydrogen towards the inlet for syngas, and to a dihydrogen outlet,
device wherein:
when the command emitted corresponds to a configuration of the reactor to promote a water gas shift reaction, the dihydrogen output selector is configured to direct the dihydrogen towards the dihydrogen outlet and the methane output selector is configured to direct the methane towards the methane recirculator; and
when the command emitted corresponds to a configuration of the reactor to promote a Sabatier reaction, the dihydrogen output selector is configured to direct the dihydrogen towards the dihydrogen recirculator, and the methane output selector is configured to direct the methane towards the methane outlet.

12. Device according to claim 1, wherein the catalytic conversion reactor is an isothermal reactor.

13. Device according to claim 1, wherein the catalytic conversion reactor.

14. Device according to claim 1, wherein there is only one catalytic conversion reactor.

15. Method for the hybrid production of synthetic dihydrogen and/or synthetic methane, comprising:
a step of selecting a configuration for operating a conversion reactor;
a step of emitting a command representative of the selected configuration;
a step of configuring the conversion reactor as a function of the command emitted according to one of the two following configurations:
a first configuration in which the operating conditions of the reactor promote a Sabatier reaction, so as to produce an outlet gas comprising mainly methane, or
a second configuration in which the operating conditions of the reactor promote a water gas shift reaction, so as to produce an outlet gas comprising mainly dihydrogen;
a step of inputting a stream of synthetic gas, (known as “syngas”);
a step of catalytic conversion reaction according to the selected configuration; and
a step of outputting a stream of synthetic dihydrogen and/or synthetic methane.
